(19) United States
(12) Patent Application Publication
(10) Pub. No.: US 2008/0175805 A1
(43) Pub. Date: Jul. 24, 2008

(54) Cosmetic foundation

(75) Inventor: Nathalie Schlemer, Los Angeles, CA (US)

Correspondence Address: Cislo & Thomas LLP, 1333 2nd Street, Suite #500, Santa Monica, CA, 90401-4110, US

(73) Assignee: Guthy-Renker Corporation

(21) Appl. No.: 11/655,420

(22) Filed: Jan. 19, 2007

(51) Int. Cl. A61K 8/97; A61Q 1/02

(52) U.S. Cl. 424/63

(57) ABSTRACT

The present invention provides a cosmetic mineral foundation, which is a loose powder, comprising Unfermented Green Rooibos Extract (Aspalathus Linearis Leaf), which functions as an antioxidant, and helps reduce the appearance of fine lines and wrinkles, and diminish signs of aging caused by free-radical damage. The foundation is gentle for all skin types, can be used daily, and provides effective anti-aging treatment as well as quality skin coverage.

### BACKGROUND

1. Field of the Invention

The present invention relates to cosmetic compositions and, in particular, to a cosmetic foundation.

2. Description of the Related Art

There is always a need in the cosmetic industry for improved products which function effectively, and improve the overall quality of the skin. Powder foundations are well known cosmetics, and have been used for many years. While foundations, which are in a liquid form, have added skin benefiting agents, such as acne control agents, wrinkle fighting agents, and moisturizers, such added skin benefiting agents are not typically found in loose powdered foundations. This may be due to the fact that most common skin benefiting agents, which provide significant skin benefit, can be formulated in a liquid medium, and not in a dry powdered form. Generally, it is well accepted in the cosmetics industry that the functionality of powdered foundations is limited to providing skin coverage and color enhancement for affecting the temporary look of the skin, and sun protection, which naturally accompanies the skin coverage. A user of a powdered foundation, seeking lasting and significant improvement in skin quality typically may have to apply at least one separate product, usually a cream or lotion, which provides the additional benefit sought. Additionally, users may apply such separate products at a time when the foundation is not worn, so as to not affect the functionality of the foundation.

### SUMMARY

The present invention provides a cosmetic mineral foundation, which is a loose powder, comprising Unfermented Green Rooibos Extract (Aspalathus Linearis Leaf) combined with zinc oxide, which is preferably micronized, boron nitride, mica, bisabolol, coloring agents preferably comprising titanium dioxide and iron oxide, and optionally bismuth oxychloride. The foundation is gentle for all skin types, can be used daily, and provides significant skin benefits including antioxidant, anti-irritant, anti-microbial, anti-allergenic, and anti-aging benefits. It further helps reduce the appearance of fine lines and wrinkles, and diminish signs of aging caused by free-radical damage, as well as quality skin coverage. As such, it provides not just color enhancement, but also benefits the skin.

### DETAILED DESCRIPTION

The detailed description set forth below is intended as a description of exemplary embodiments and is not intended to represent the only forms in which the exemplary embodiments may be constructed and/or utilized. The description sets forth the functions and the sequence of steps for constructing and/or operating the exemplary embodiments. However, it is to be understood that the same or equivalent functions and sequences may be accomplished by different exemplary methods are also intended to be encompassed within the spirit and scope of the invention.

As used herein, “safe and effective amount” means a sufficient amount of a compound, composition or other material described by this phrase to significantly induce a positive modification in the condition being treated, but low enough to avoid undue side effects (e.g., significant skin irritation or sensitization), within the scope of sound judgment of the skilled person. The safe and effective amount of the compound, composition or other material may vary with the particular skin being treated, the age and physical condition of the biological subject being treated, the severity of the condition, the duration of treatment, the nature of concurrent therapy, the specific compound, composition, or other material employed, the particular cosmetically acceptable topical carrier utilized, and the factors within the knowledge and expertise of the skilled person.

The present invention provides a cosmetic foundation, which is a mineral loose powder, comprising Unfermented Green Rooibos Extract (Aspalathus Linearis Leaf) combined with zinc oxide, which is preferably micronized, boron nitride, mica, bisabolol, coloring agents preferably comprising titanium dioxide and iron oxide, and optionally bismuth oxychloride. The foundation is gentle for all skin types, can be used daily, and provides significant skin benefits including antioxidant, anti-irritant, anti-microbial, anti-allergenic, and anti-aging benefits. It further helps reduce the appearance of fine lines and wrinkles, and diminish signs of aging caused by free-radical damage, as well as quality skin coverage.

Green Rooibos Extract is a natural botanical having anti-oxidant, anti-inflammatory/anti-irritant, and free radical scavenging properties, partly due to its unique flavonoid content which includes Aspalathin. Additionally, it has high anti-allergenic and anti-microbial effects and further prevents eczema. Its properties are ascribed to the high level of antioxidants, minerals, and polyphenols. While its use in cosmetics is generally known, it is much more commonly used and known as an anti-oxidant tea, where it is typically used in its fermented form. The fermentation process normally starts naturally immediately after the plant is harvested. However, fermentation alters and reduces the flavonoid composition hence significantly reducing the anti-oxidant activity. The antioxidant activity of the unfermented powdered extract has been found to be much higher than the usual rooibos extract which is fermented. The mineral powder foundation of the present invention includes a safe and effective amount of Unfermented Green Rooibos Extract (Aspalathus Linearis Leaf), preferably at a concentration of approximately around up to 1% by weight, and more preferably at about 0.1% by weight.

The zinc oxide used in the present formulation is preferably micronized, having a particle size of preferably less than 0.2 microns, and more preferably, about less than 0.1 microns, which increase its sunscreening effectiveness and esthetics, compared to larger particles. The formulation of the present invention preferably uses zinc oxide at a concentration of roughly about 9-15% by weight, and more preferably at about 12%.

The formulation of the present invention further includes boron nitride, which is a slip agent, at a concentration of approximately 9-15% by weight, and more preferably at around 1%. The formulation further includes bisabolol, as a skin soothing agent, at a concentration of approximately 0.01-1% by weight, and more preferably at around 0.1%. The formulation further contains visual effect agents comprising mica, and colorants, which are preferably mineral colorants such as titanium dioxide and iron oxides. The concentrations of the visual effect agents will depend on the color shades and visual effects desired, and the particular agents used. For the particular visual affect agents in Examples 1-6 below, which include mica, zinc oxide, and iron oxide, the combined concentrations may vary, between roughly about 40-85% by weight of total composition. Although it is preferred to use mineral colorants, one skilled in the art may be able to use artificial colors to create a similar formulation. The formulation of the present invention also may optionally include bismuth oxychloride, as a skin adhesion agent, at concentrations of approximately between 25-35%, and more preferably at around 31%.

The foundation of the present invention is preferably made of natural botanicals and minerals, and preferably contains no preservatives, fragrances, dyes or chemicals, and oils, and is preferably formulated without fillers such as silicone, talc, and mineral oil. It is especially formulated for troubled and sensitive skin. The inert minerals do not support bacterial growth. The natural pigments do not pile on the edges of skin pores, which would make pores more visible. Additionally, the formulation is designed to deflect light away from skin imperfections, for a line minimizing effect.

### EXAMPLE 1

The following is an example for the preparation of a mineral foundation in having an “Almond” colored shade:

| SEQ. | TRADE NAME | INCI Name | % Wt | Supplier |
|---|---|---|---|---|
| (A) | Z-Cote ® | Zinc Oxide | 12.000 | BASF |
| (A) | Atlas White 28 | Titanium | 16.000 | LCW |
| | 190 Oil | Dioxide (Cl | | |
| | Dispersible Lot# | 778891) | | |
| | W05280 | | | |
| (A) | Unipure Red | Iron Oxide (Cl | 2.130 | LCW |
| | LC381 Lot# | 77491) | | |
| | 87259 | | | |
| (A) | Unipure Yellow | Iron Oxide (CI | 9.50 | LCW |
| | LC181 Lot# | 77492) | | |
| | B1948 | | | |
| (A) | Unipure Black | Iron Oxide (CI | 1.53 | LCW |
| | LC989 Lot# | 77499) | | |
| | B1273 | | | |
| (A) | Boron Nitride | Boron Nitride | 1.00 | GE Adv Ceramincs |
| | Powder CC6058 | | | |
| (A) | Lipomic 601 | Mica | 9.22 | Lipo Chemicals |
| (A) | Sericite PHN | Mica | 15.42 | Presperse |
| (A) | Mearite LBU | Bismuth | 31.00 | Engelhard |
| | | Oxychloride | | |
| | | (CI 77163) | | |
| (B) | Sericite PHN | Mica | 2.0 | Presperse |
| (B) | Alpha-Bisabolol | Bisabolol | 0.10 | BASF |
| | Natural | | | |
| (B) | Unfermented | Aspalathus | 0.10 | Jarchem |
| | Green Roolbos | Linearis Leaf | | |
| | Extract | Extract | | |

### EXAMPLE 2

The following is an example for the preparation of a mineral foundation having a “Buff” colored shade:

| SEQ. | TRADE NAME | INCI Name | % Wt | Supplier |
|---|---|---|---|---|
| (A) | Z-Cote ® | Zinc Oxide | 12.000 | BASF |
| (A) | Atlas White 28 | Titanium | 25.500 | LCW |
| | 190 Oil | Dioxide (Cl | | |
| | Dispersible Lot# | 778891) | | |
| | W05280 | | | |
| (A) | Unipure Red | Iron Oxide (Cl | 0.960 | LCW |
| | LC381 Lot# | 77491) | | |
| | 87259 | | | |
| (A) | Unipure Yellow | Iron Oxide (CI | 5.000 | LCW |
| | LC181 Lot# | 77492) | | |
| | B1948 | | | |
| (A) | Unipure Black | Iron Oxide (CI | 0.300 | LCW |
| | LC989 Lot# | 77499) | | |
| | B1273 | | | |
| (A) | Boron Nitride | Boron Nitride | 1.000 | GE Adv Ceramincs |
| | Powder CC6058 | | | |
| (A) | Lipomic 601 | Mica | 9.220 | Lipo Chemicals |
| (A) | Sericite PHN | Mica | 12.820 | Presperse |
| (A) | Mearite LBU | Bismuth | 31.000 | Engelhard |
| | | Oxychloride | | |
| | | (CI 77163) | | |
| (B) | Sericite PHN | Mica | 2.000 | Presperse |
| (B) | Alpha-Bisabolol | Bisabolol | 0.100 | BASF |
| | Natural | | | |
| (B) | Unfermented | Aspalathus | 0.100 | Jarchem |
| | Green Roolbos | Linearis Leaf | | |
| | Extract | Extract | | |

### EXAMPLE 3

The following is an example for the preparation of a mineral foundation having a “Latte” colored shade:

| SEQ. | TRADE NAME | INCI Name | % Wt | Supplier |
|---|---|---|---|---|
| (A) | Z-Cote ® | Zinc Oxide | 12.000 | BASF |
| (A) | Atlas White 28 | Titanium | 37.000 | LCW |
| | 190 Oil | Dioxide (Cl | | |
| | Dispersible Lot# | 778891) | | |
| | W05280 | | | |
| (A) | Unipure Red | Iron Oxide (Cl | 1.500 | LCW |
| | LC381 Lot# | 77491) | | |
| | 87259 | | | |
| (A) | Unipure Yellow | Iron Oxide (CI | 5.000 | LCW |
| | LC181 Lot# | 77492) | | |
| | B1948 | | | |
| (A) | Unipure Black | Iron Oxide (CI | 0.600 | LCW |
| | LC989 Lot# | 77499) | | |
| | B1273 | | | |
| (A) | Boron Nitride | Boron Nitride | 1.000 | GE Adv Ceramincs |
| | Powder CC6058 | | | |
| (A) | Lipomic 601 | Mica | 9.220 | Lipo Chemicals |
| (A) | Sericite PHN | Mica | 0.480 | Presperse |
| (A) | Mearite LBU | Bismuth | 31.000 | Engelhard |
| | | Oxychloride | | |
| | | (CI 77163) | | |
| (B) | Sericite PHN | Mica | 2.000 | Presperse |
| (B) | Alpha-Bisabolol | Bisabolol | 0.100 | BASF |
| | Natural | | | |
| (B) | Unfermented | Aspalathus | 0.100 | Jarchem |
| | Green Roolbos | Linearis Leaf | | |
| | Extract | Extract | | |

### EXAMPLE 4

The following is an example for the preparation of a mineral foundation having a “Golden” colored shade:

| SEQ. | TRADE NAME | INCI Name | % Wt | Supplier |
|---|---|---|---|---|
| (A) | Z-Cote ® | Zinc Oxide | 12.000 | BASF |
| (A) | Atlas White 28 | Titanium | 21.000 | LCW |
| | 190 Oil | Dioxide (Cl | | |
| | Dispersible Lot# | 778891) | | |
| | W05280 | | | |
| (A) | Unipure Red | Iron Oxide (Cl | 0.750 | LCW |
| | LC381 Lot# | 77491) | | |
| | 87259 | | | |
| (A) | Unipure Yellow | Iron Oxide (CI | 6.000 | LCW |
| | LC181 Lot# | 77492) | | |
| | B1948 | | | |
| (A) | Unipure Black | Iron Oxide (CI | 0.240 | LCW |
| | LC989 Lot# | 77499) | | |
| | B1273 | | | |
| (A) | Boron Nitride | Boron Nitride | 1.000 | GE Adv Ceramincs |
| | Powder CC6058 | | | |
| (A) | Lipomic 601 | Mica | 9.220 | Lipo Chemicals |
| (A) | Sericite PHN | Mica | 16.590 | Presperse |
| (A) | Mearite LBU | Bismuth | 31.000 | Engelhard |
| | | Oxychloride | | |
| | | (CI 77163) | | |
| (B) | Sericite PHN | Mica | 2.000 | Presperse |
| (B) | Alpha-Bisabolol | Bisabolol | 0.100 | BASF |
| | Natural | | | |
| (B) | Unfermented | Aspalathus | 0.100 | Jarchem |
| | Green Roolbos | Linearis Leaf | | |
| | Extract | Extract | | |

### EXAMPLE 5

The following is an example for the preparation of a mineral foundation having a “Mocha” colored shade:

| SEQ. | TRADE NAME | INCI Name | % Wt | Supplier |
|---|---|---|---|---|
| (A) | Z-Cote ® | Zinc Oxide | 12.000 | BASF |
| (A) | Atlas White 28 | Titanium | 16.000 | LCW |
| | 190 Oil | Dioxide (Cl | | |
| | Dispersible Lot# | 778891) | | |
| | W05280 | | | |
| (A) | Unipure Red | Iron Oxide (Cl | 5.480 | LCW |
| | LC381 Lot# | 77491) | | |
| | 87259 | | | |
| (A) | Unipure Yellow | Iron Oxide (CI | 16.000 | LCW |
| | LC181 Lot# | 77492) | | |
| | B1948 | | | |
| (A) | Unipure Black | Iron Oxide (CI | 2.162 | LCW |
| | LC989 Lot# | 77499) | | |
| | B1273 | | | |
| (A) | Boron Nitride | Boron Nitride | 1.000 | GE Adv Ceramincs |
| | Powder CC6058 | | | |
| (A) | Lipomic 601 | Mica | 9.220 | Lipo Chemicals |
| (A) | Sericite PHN | Mica | 5.958 | Presperse |
| (A) | Mearite LBU | Bismuth | 31.000 | Engelhard |
| | | Oxychloride | | |
| | | (CI 77163) | | |
| (B) | Sericite PHN | Mica | 2.000 | Presperse |
| (B) | Alpha-Bisabolol | Bisabolol | 0.100 | BASF |
| | Natural | | | |
| (B) | Unfermented | Aspalathus | 0.100 | Jarchem |
| | Green Roolbos | Linearis Leaf | | |
| | Extract | Extract | | |

### EXAMPLE 6

The following is an example for the preparation of a mineral foundation having a “Nude” colored shade:

| SEQ. | TRADE NAME | INCI Name | % Wt | Supplier |
|---|---|---|---|---|
| (A) | Z-Cote ® | Zinc Oxide | 12.000 | BASF |
| (A) | Atlas White 28 | Titanium | 28.000 | LCW |
| | 190 Oil | Dioxide (Cl | | |
| | Dispersible Lot# | 778891) | | |
| | W05280 | | | |
| (A) | Unipure Red | Iron Oxide (Cl | 1.490 | LCW |
| | LC381 Lot# | 77491) | | |
| | 87259 | | | |
| (A) | Unipure Yellow | Iron Oxide (CI | 3.610 | LCW |
| | LC181 Lot# | 77492) | | |
| | B1948 | | | |
| (A) | Unipure Black | Iron Oxide (CI | 0.500 | LCW |
| | LC989 Lot# | 77499) | | |
| | B1273 | | | |
| (A) | Boron Nitride | Boron Nitride | 1.000 | GE Adv Ceramincs |
| | Powder CC6058 | | | |
| (A) | Lipomic 601 | Mica | 9.220 | Lipo Chemicals |
| (A) | Sericite PHN | Mica | 10.980 | Presperse |
| (A) | Mearite LBU | Bismuth | 31.000 | Engelhard |
| | | Oxychloride | | |
| | | (CI 77163) | | |
| (B) | Sericite PHN | Mica | 2.000 | Presperse |
| (B) | Alpha-Bisabolol | Bisabolol | 0.100 | BASF |
| | Natural | | | |
| (B) | Unfermented | Aspalathus | 0.100 | Jarchem |
| | Green Roolbos | Linearis Leaf | | |
| | Extract | Extract | | |

In each of Examples 1-6, blend all ingredients of Seq. A in a suitable blender. When the mixture is uniform, grind or pulverize through a 0.010″ Herringbone screen. Check the uniformity and fineness of the grind by pressing a sample between 2 pieces of white filter paper. If the batch is uniform and free of pigment specks, return the batch to the blender and continue mixing. Premix Seq. B materials in a separate container and disperse until the phase is completely uniform. Add the Seq. B mix to the Seq. A batch and mix until the batch is completely uniform and homogeneous. Pulverize the entire batch through a 0.250″ screen as a finishing step. Mix the batch until uniform. Store the batch in airtight containers until ready for filling.

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the description is illustrative and not meant to be a limitation thereof.

1-9. (canceled)

10. A cosmetic foundation comprising:
approximately 0.1% by weight of total composition of unfermented green rooibos extract;
approximately 12% by weight of total composition of micronized zinc oxide;
approximately 1% by weight of total composition of boron nitride;
approximately 31% by weight of total composition of bismuth oxychloride;
approximately 0.1% by weight of total composition of bisabolol; and
approximately between 55% and 57% by weight of total composition of visual effect and coloring agents comprising a combination of titanium dioxide, iron oxides, and micas, wherein
said foundation is free of preservatives, fragrances, and oils.

11. A cosmetic foundation comprising:
approximately 0.1% by weight of total composition of unfermented green rooibos extract;
approximately 12% by weight of total composition of micronized zinc oxide;
approximately 1% by weight of total composition of boron nitride;
approximately 31% by weight of total composition of bismuth oxychloride;
approximately 0.1% by weight of total composition of bisabolol; and
at least approximately 15% by weight of total composition of titanium dioxide;
at least approximately 5% by weight of total composition of iron oxides; and
at least approximately 10% by weight of total composition of mica.

12. A cosmetic foundation comprising:
unfermented green rooibos extract, at a concentration of approximately 0.01 to 1% by weight of total composition;
micronized zinc oxide, at a concentration of approximately 9-15% by weight of total composition;
optionally, bismuth oxychloride, at a concentration of approximately 25-35% by weight of total composition;
bisabolol, at a concentration of approximately 0.01 to 1% by weight of total composition;
boron nitride, at a concentration of approximately 0.1 to 2% by weight of total composition;
and
visual effect and coloring agents comprising titanium dioxide, iron oxide, mica, or a combination thereof, at a concentration of approximately 40-85% by weight of total composition wherein
said foundation is effective in protecting the skin against free radical damage and further acts as an anti-inflammatory agent.

13. The composition of claim 12 wherein the concentration of said rooibos extract is approximately 0.1%.

14. The composition of claim 12 wherein said foundation is free of preservatives, fragrances, and oils.

15. The composition of claim 12 wherein said foundation is a loose powder.

16. A cosmetic foundation comprising:
green rooibos extract, zinc oxide, bismuth oxychloride, bisabolol, boron nitride, titanium dioxide, iron oxide, and mica, wherein
said foundation is a loose powder which is effective in protecting the skin against signs of aging caused by free-radical damage, and is further has anti-irritant, anti-microbial, and anti-allergenic skin benefiting properties.

17. The cosmetic foundation of claim 16, wherein
said foundation is made of natural botanicals and minerals, and
said foundation is preservative-free, fragrance-free, and oil-free.

18. A method of providing skin coverage, while, at the same time, providing skin treatment against signs of aging, comprising applying a powder foundation to the skin, said powder foundation comprising:
green rooibos extract,
zinc oxide,
optionally, bismuth oxychloride,
bisabolol,
boron nitride, and
visual effect agents comprising iron oxide, titanium dioxide, mica, or a combination thereof, wherein
said rooibos extract is effective in protecting the skin against free radical damage and further acts as an anti-inflammatory agent.

19. The method of claim 18 wherein said green rooibos extract is unfermented, and wherein said zinc oxide is micronized.

20. The method of claim 18 wherein said powder foundation comprises:
unfermented green rooibos extract, at a concentration of approximately 0.01 to 1% by weight of total composition;
zinc oxide, at a concentration of approximately 9-15% by weight of total composition;
optionally, bismuth oxychloride, at a concentration of approximately 25-35% by weight of total composition;
bisabolol, at a concentration of approximately 0.01 to 1% by weight of total composition;
boron nitride, at a concentration of approximately 0.1 to 2% by weight of total composition; and
said visual effect agents, at a concentration of approximately 40-85% by weight of total composition.

21. A cosmetic foundation comprising:
unfermented Green Rooibos Extract;
micronized zinc oxide; and
at least two ingredients selected from the group comprising boron nitride, mica, bisabolol, titanium dioxide, iron oxide, and bismuth oxychloride, wherein
said foundation is a loose powder, and provides antioxidant, anti-irritant, anti-microbial, anti-allergenic, and anti-aging benefits to the skin.

22. The foundation of claim 21, wherein
said foundation is made of natural botanicals and minerals only, and is further free of preservatives, fragrances, and oils.

23. The foundation of claim 21, wherein said foundation comprises:
at least 0.01% by weight of total composition of said Rooibos extract; and
at least 9% by weight of total composition of said zinc oxide.

24. The foundation of claim 21, wherein said foundation is preservative-free, fragrance-free, and oil-free.
